(19) United States
(12) Patent Application Publication
(10) Pub. No.: US 2013/0267591 A1
(43) Pub. Date: Oct. 10, 2013

(54) NOVEL OPHTHALMIC COMPOSITIONS

(71) Applicants: Ajay Jaysingh Khopade, Baroda (IN); Arindam Halder, Baroda (IN); Subhas Balaram Bhowmick, Baroda (IN)

(72) Inventors: Ajay Jaysingh Khopade, Baroda (IN); Arindam Halder, Baroda (IN); Subhas Balaram Bhowmick, Baroda (IN)

(73) Assignee: SUN PHARMA ADVANCED RESEARCH COMPANY LIMITED, Mumbai, Maharashtra (IN)

(21) Appl. No.: 13/696,685

(22) PCT Filed: May 6, 2011
(86) PCT No.: PCT/IN2011/000320

§ 371 Date: Jun. 21, 2013

(30) Foreign Application Priority Data
May 7, 2010 (IN) 1448/MUM/2010

(51) Int. Cl. A61K 31/216; A61K 45/06

(52) U.S. Cl. CPC A61K 31/216; A61K 45/06
USPC 514/530

(57) ABSTRACT

An ophthalmic solution comprising therapeutically effective amount of a prostaglandin or its analog and water soluble excipient(s) dissolved in a pharmaceutically acceptable vehicle, wherein the solution is free of a surfactant.

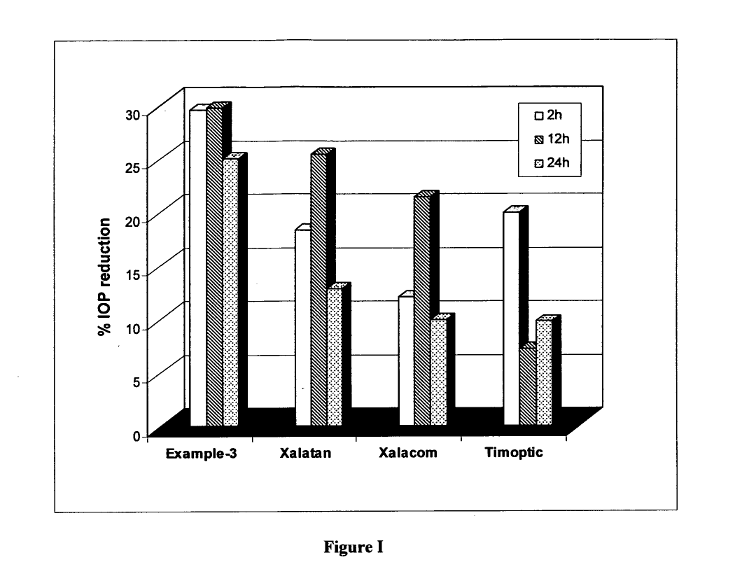

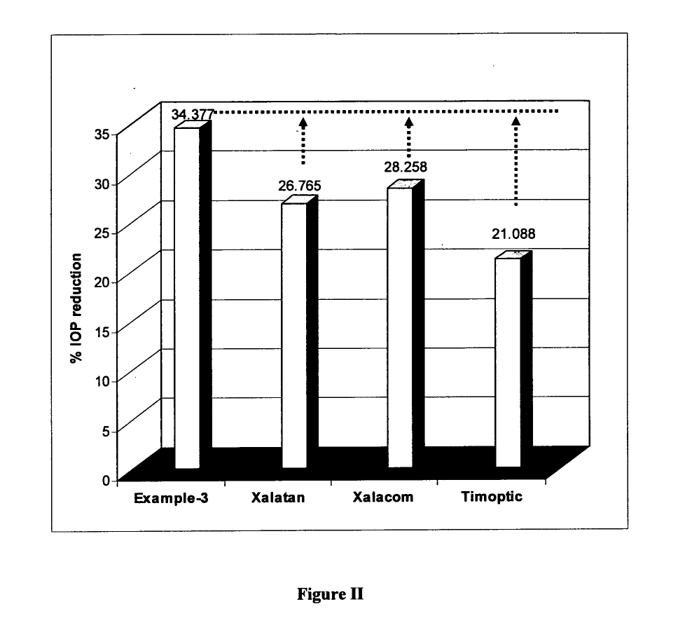

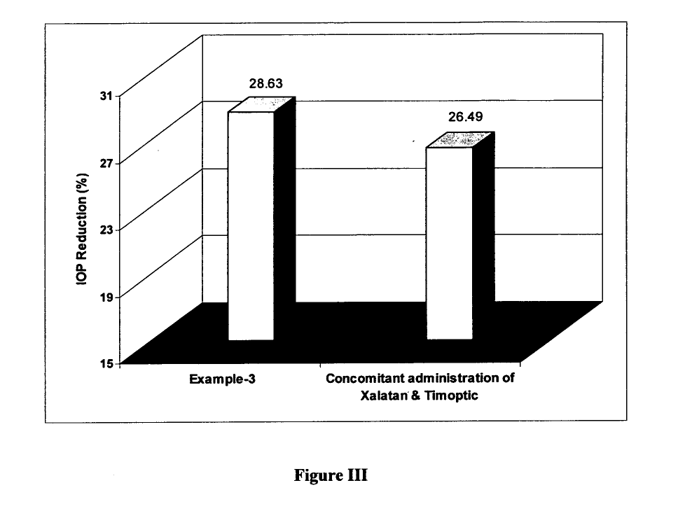

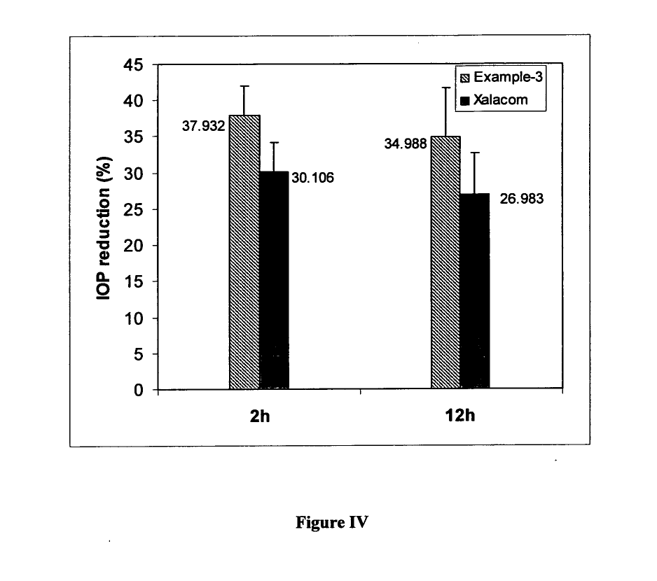

The present invention relates to a novel ophthalmic solution prostaglandin or its analogs alone or in combination with other antiglaucoma agents.

### BACKGROUND OF THE INVENTION

Prostaglandins are well known active substances administered to humans or animals via the topical route in the form of ophthalmic solutions for the treatment of glaucoma. The prostaglandins may also be used in combination with a second anti-glaucoma agent such as a beta-blocker, a carbonic anhydrase inhibitor or an alpha-adrenergic agonist.

Prostaglandin or its analogs, particularly the ester derivatives such as latanoprost, travoprost or the amide derivatives such as bimatoprost have notoriously low water solubility. The use of compounds which exert a surfactant like activity in to solubilize them is therefore, very common. Currently available prostaglandin ophthalmic solution, are found to contain a typical surfactant or a quaternary ammonium salt which is known to have a surfactant like activity apart from preservative property. Representative examples of typical surfactants incorporated in the ophthalmic solutions of prostaglandin analogs alone or in combination with other antiglaucoma agent, like for example, beta adrenergic blocking agent or alpha adrenergic blocking agent or any other active agent, are tabulated here:

| Product | Active Ingredient | Surfactant |
|---|---|---|
| Xalatan ® | Latanoprost | Benzalkonium chloride |
| Travatan Z ® | Travoprost | polyoxyl 40 hydrogenated |
| | | castor oil (Cremphore) |
| Xalacom ® | Latanoprost and timolol | Benzalkonium chloride |
| Lumigan ® | Bimatoprost | Benzalkonium chloride |
| Ganfort ® | Bimatoprost and timolol | Benzalkonium chloride |
| Duotrav ® | Travoprost and timolol | Benzalkonium chloride |
| Rescula ® | Unoprostone isopropyl | Polyoxyethylene-20-sorbitan- |
| | | monooleate |

Apart from the approved products, the patent literature also represents numerous efforts of solubilizing prostaglandins with the help of solubilizers such as polyoxyethylene-20-sorbitan-monooleate, polyoxy stearates like Solutol® with or without other antiglaucoma agent like beta adrenergic blocking agent. Below is a list of patent documents that disclose the use of surfactant in a prostaglandin ophthalmic solution alone or in combination with other antiglaucoma agent.

| Product disclosed in | | |
|---|---|---|
| Literature | Prostaglandin | Surfactant |
| U.S. Pat. No. 7,074,827 | Latanoprost | Polyoxyethylene-20-sorbitan- |
| | | monooleate |
| US20100201720 | Prostaglandin | Solutol |
| WO/2009/145356 | Tafluprost | Polyoxyethylene-20-sorbitan- |
| | | monooleate |
| US20030018079 | Latanoprost | Polyoxyethylene-20-sorbitan- |
| | and Timolol | monooleate and |
| | | Benzalkonium chloride |

Generally, the formulation development of ophthalmic solution of prostaglandin or their combination with other active ingredient, over the years, is directed towards achieving a stable composition particularly in view of the fact that prostaglandins are also known to chemically unstable. Further, the literature provides evidences that the prostaglandins were associated with an adsorption problem to the poly-ethylene multidose containers. Some solutions to solve these problems are described in patent documents such as, for example, U.S. Pat. No. 6,235,781 which discloses that use of a surfactant to prevent the adsorption of prostaglandin analogues on to the plastic containers. The inventor of the present invention also faced and tackled this problem of adsorption of prostaglandin as described in WO 2009/084021. It was found out by inventors that a micro-emulsion formulation of prostaglandin containing polyoxy hydroxystearate (commonly known as Solutol HS) provides the solution to stability problem associated with adsorption. Another patent application, namely, United States Patent number US 20090234013A1, discloses a solution which include a therapeutic agent and a relatively low amount of surfactant for providing higher bioavailability of prostaglandin such as travoprost. Thus, this prior art as well teaches to include some amount of a surfactant such as ethoxylated and/or hydrogenated vegetable oil. This implies that the surfactant is always desirable to make the solution however it is preferable to keep it as low as possible.

Now, the inventors surprisingly and unexpectedly found that the prostaglandin analogs can be effectively formulated into an ophthalmic solution vehicle having a water soluble excipient(s) dissolved in the vehicle, wherein the ophthalmic solution is free of a surfactant. When the efficacy of the ophthalmic solution was compared with an ophthalmic solution comprising a surfactant, it was found that the ophthalmic solution provided equivalent or improved efficacy in reducing the intraocular pressure. Particularly, the ophthalmic solution of present invention was found to provide equivalent efficacy at half the dose compared to the marketed product available under the tradename of Xalatan® when tested in animals. This achievement of equivalent efficacy at half the dose of latanoprost was indeed unexpected and surprising. It was further found that the % intraocular pressure reduction at 12 hour time point, which apparently provides a peak IOP reduction was higher compared to the % intraocular pressure reduction at 12 hours, for Xalatan® which is a latanoprost ophthalmic solution having benzalkonium chloride as a surfactant. This effect of improved efficacy inspite of the absence of a surfactant, was also observed when the ophthalmic solution of the present invention was made of a prostaglandin or its analog and another antiglaucoma agent like a beta adrenergic blocking agent. The ophthalmic composition comprising prostaglandin or its nalog and a beta-adrenergic blocking agent that is free of surfactant, the composition remained stable and did not show any hazyness. The composition was clear on storage and was chemically stable. Thus, the invention not only provided a physically stable composition comprising the two active ingredients, but also provided an ophthalmic composition that was more efficacious. Since the compositions are intended for ophthalmic purposes, it is always desirable that the compositions are devoid of excessive additives. Therefore, the present invention can be said to achieve not only the patient compliance but also achieved an improved efficacious composition.

Thus, the ophthalmic composition of the present invention comprises a combination of a prostaglandin and a beta-adrenergic blocking agent, characterized in that it does not use any surfactant or a surfactant preservative in a concentration that acts as a solubilizer such as those from alkyl quaternary ammonium surfactant like benzalkonium chloride, benzdodecinium chloride and like and mixtures thereof. In one preferred embodiment, the ophthalmic composition includes a vehicle that is free of surfactants and added preservatives and is able to provide a beta-adrenergic blocking agent when administered topically such that effect is sustained for 24 hours, that is the ophthalmic composition is said to be suitable for once-a-day administration. Therefore, one of the embodiment of the present invention can be said to provide an ophthalmic composition comprising latanoprost and once-a-day composition of a beta-adrenergic blocking agent, wherein the composition is free of surfactant and optionally, free of added preservative and is found to be suitable for treating the affected eye of a glaucoma patient.

The ophthalmic solution of the present invention is free of a surfactant as well as free of anti-microbial preservatives defined by the class of quaternary ammonium compounds, organo-mercurials and substituted alcohol and phenols. It is known that these antimicrobials are often toxic to the sensitive tissues of the eye. The present invention thereofore fulfils the need of an ophthalmic solution which is stable as well having improved efficacy while not compromising on the antimicrobial activity. The present invention provides an ophthalmic solution comprising prostaglandins which obtains dual benefits of improved efficacy and avoidance of undesirable effects of the preservatives.

### OBJECTS OF THE INVENTION

The object of the invention is to provide an ophthalmic solution that allows dose reduction of the prostaglandin while achieving equivalent efficacy.

The present invention relates to an ophthalmic solution comprising therapeutically effective amount of a prostaglandin analogue and another active ingredient, wherein the solution provides therapeutic effect sustaining for 24 hours i.e. to provide a once-a-day therapy.

The object of the present invention to provide a stable ophthalmic solution of prostaglandin analogs.

The object of the present invention to provide a stable ophthalmic solution of prostaglandin analogs and beta adrenergic active agents.

### SUMMARY OF THE INVENTION

Thus, the present invention provides an ophthalmic solution comprising prostaglandins which obtains dual benefit of improved efficacy and avoidance of undesirable effects of the preservatives. The ophthalmic solution of the present invention is free of a surfactant as well as free of anti-microbial preservatives defined by the class of quaternary ammonium compounds, organo-mercurials, and substituted alcohol and phenols, It is known that these antimicrobials are often toxic to the sensitive tissues of the eye. A need therefore exists for ophthalmic solutions which have a stability, efficacy, but whose antimicrobial efficacy is not compromised.

The present invention provides an ophthalmic solution comprising therapeutically effective amount of a prostaglandin or its analog and optionally, one or more other therapeutic agents and water soluble excipient(s) dissolved in a pharmaceutically acceptable vehicle, wherein the solution is free of a surfactant.

The present invention also provides a method of treating glaucoma or ocular hypertension which comprises topically administering to an affected eye an ophthalmic solution comprising therapeutically effective amount of a prostaglandin or its analog and optionally, one or more other therapeutic agents and water soluble excipient(s) dissolved in a pharmaceutically acceptable vehicle, wherein the solution is free of a surfactant.

### BRIEF DESCRIPTION OF FIGURE

FIG. I: A comparative % reduction in the intraocular pressure of the dogs within 24 hours when the ophthalmic solution of the present invention was administered and % reduction in the intraocular pressure after the administration of already available marketed products like Xalatan®, Xalacom®, Timoptic®. It was found that the ophthalmic solution of example 3 provided a 29.43% IOP reduction at 2 hr compared to 18.19% IOP reduction when Xalatan® was administered or 12.02% IOP reduction when Xalacom® was administered or 19.82% IOP reduction when Timoptic® was administered. Similarly, example 3 provided a 29.67% IOP reduction at 12 hr compared to 25.31% IOP reduction when Xalatan® was administered or 21.28% IOP reduction when Xalacom® was administered or 7.16% IOP reduction when Timoptic® was administered. Similarly, example 3 provided a 24.87% IOP reduction at 24 hr compared to 12.77% IOP reduction when Xalatan® was administered or 9.84% IOP reduction when Xalacom® was administered or 9.72% IOP reduction when Timoptic® was administered.

FIG. II: A comparative % mean reduction in the intraocular pressure of the affected eye of dogs when the solution of the present invention was administered Vs % mean reduction in the intraocular pressure after the administration of marketed reference products such as like Xalatan®, Xalacom®, Timoptic®. The % mean reduction of the intraocular pressure was found to be higher compared to the marketed product which either contains a beta-adrenergic blocking agent such as Timoptic® or a Xalatan® which alone or their combination (Xalacom®). It was found that the mean intraocular pressure reduction achieved by administration of the ophthalmic solution of Example 3, was 34.377% compared to 26.765% achieved by Xalatan® or 28.258% achieved by Xalacom® or 21.088% achieved by Timoptic® alone.

FIG. III: It is a graph of comparison % IOP reduction when the ophthalmic solution of the present invention was administered, with % IOP reduction after the concomitant administration of marketed latanoprost and timolol products like Xalatan® and Timoptic® to the dogs It was found that the overall, mean intraocular pressure reduction achieved by the ophthalmic solution of the present invention administered once a day was 28.63% compared to 26.49% which was achieved by the concomitant administration of the marketed product of latanoprost (once a day) and timolol (twice a day) present alone in the products.

FIG. IV: A comparative % mean reduction in the intraocular pressure of the affected eye of dogs when the solution of the present invention Example 3 was administered Vs % mean reduction in the intraocular pressure after the administration of marketed reference products Xalacom® over 2 h and 12 h which represent the peak effect of Timolol and latanoprost, respectively. It is noted that the solution of example 3 has a significantly higher IOP reductions at both time points. At 2 h p=0.0054, p<0.01, at 12 h p=0.0019, p<0.01.

### DETAILED DESCRIPTION OF THE INVENTION

The term ‘surfactant’ as used herein means an amphiphilic compound that has the following properties
It has hydrophobic groups and hydrophilic groups
Can form micelles
Capable of migrating to the water surface, where the insoluble hydrophobic alkyl chains may extend out of the bulk water phase, either into the air or, if water is mixed with oil, into the oil phase, while the water soluble head group remains in the aqueous phase.
Can solubilize water insoluble substances through micellar solubilization.

The ophthalmic solutions of the present invention are characterized as being clear aqueous solution. These “solution” as stated herein, are defined as those solutions which do not cause any visual disturbance and/or do not affect vision, upon topical instillation to the eye and when examined under suitable conditions of visibility, are practically clear and practically free from particles. Ophthalmic solutions containing polymers which show percent transmission greater than 90% are referred to as ‘solution’. When light is allowed to pass through the ophthalmic solution of the present invention, the percentage of incident light which is transmitted through the solution is referred to as “Percent Transmission”. The clarity of the solution is poor if percent transmission is less than 85%. Preferably the percent transmission is greater than 90%. Generally, the percent transmission is determined at a wavelength of about 650 nm, but any other suitable wavelength may be selected for determining the clarity of the solution.

The prostaglandin or its analog used in the ophthalmic solution of the present invention includes, but are not limited to, all pharmaceutically acceptable prostaglandins, their derivatives and analogs, and their pharmaceutically acceptable esters and salts (hereinafter collectively referred to as “prostaglandins” or “PG's”), which are useful for reducing intraocular pressure when applied topically to the eye. Such prostaglandins include the natural compounds, such as for example PGE₁, PGE₂, PGE₃, PGD₂, PGF_{1α}, PGF_{2α}, PGF_{3α}, PGI₂ (prostacyclin), as well as analogs and derivatives of these compounds which are known to have similar biological activities of either greater or lesser potencies. Analogs of the natural prostaglandins include but are not limited to: alkyl substitutions (e.g., 15-methyl or 16,16-dimethyl), which confer enhanced or sustained potency by reducing biological metabolism or alter selectivity of action; saturation (e.g. 13,14-dihydro) or unsaturation (e.g., 2,3-didehydro, 13,14-didehydro), which confer sustained potency by reducing biological metabolism or alter selectivity of action; deletions or replacements (e.g. 11-deoxy, 9-deoxo-9-methylene), which enhance chemical stability and/or selectivity of action; and omega chain modifications (e.g., 18,19,20-trinor-17-phenyl, or 17,18,19,20-tetranor-16-phenoxy), which enhance selectivity of action and reduced biological metabolism.

Derivatives of these prostaglandins that may be formulated in the solution of the present invention include all pharmaceutically acceptable esters or amides, which may be attached to the 1-carboxyl group or any of the hydroxyl groups of the prostaglandin by use of the corresponding alcohol or organic acid reagent, as appropriate. The terms “analogs” and “derivatives” include compounds which exhibit functional and physical responses similar to those of prostaglandins per se. Prostaglandins are well known in the art. Particular prostaglandins that may be formulated in the solutions of the present invention include for example trimoprostil, rioprostil, cloprostenol, fluprostenol, luprostiol, etiproston, tiaprost, latanoprost, travoprost, bimatoprost, tafluprost, unoprostone and its derivatives like unoprostone isopropyl, misoprostol, sulfoprostone, gemeprost, alfaprostol, delprostenate, and the like. Pharmaceutical solutions of the present invention include one or more prostaglandins as described above in an amount between about 0.0001% w/v and about 0.2% w/v. The presently preferred amount of prostaglandin or its derivative is from about 0.001% to 0.05%, preferably about 0.0015% to about 0.03%.

In one embodiment, the ophthalmic solution of the present invention is free of surfactant and preservative as well as free of any cyclodextrin which solubilizes the prostaglandins by inclusion complexes. The ophthalmic solutions disclosed in patent application EP0435682 A2 uses cyclodextrin to solubilize the TRIS derivatives of the prostaglandins. This patent also teaches to include one or more preservatives.

In one embodiment of the present invention, latanoprost which is a prostaglandin F2α analogue, namely isopropyl-(Z)-7[(1R,2R,3R,5S)3,5-dihydroxy-2-[(3R)-3-hydroxy-5-phenylpentyl]cyclopentyl]-5-heptenoate is used. It may be present in an amount ranging from about 0.0001% w/v to about 0.2% w/v. Preferably, latanoprost is used in amounts of about 0.005% w/v. In another embodiment, travoprost is used as the prostaglandin derivative in amounts ranging from about 0.0001% w/v to about 0.2% w/v preferably in an amount 0.004% w/v. In yet another embodiment, bimatoprost is used as the prostaglandin derivative in amounts ranging from about 0.0001% w/v to about 0.2% w/v, preferably in an amount 0.03% w/v. In yet another embodiment tafluprost is used in amounts ranging from about 0.0001% w/v to about 0.2% w/v, preferably in an amount 0.0015% w/v.

In one preferred embodiment of the present invention, the ophthalmic solution is free of surfactant as well as free of a preservative or antimicrobial preservatives defined by the class of quaternary ammonium compounds, organic mercurial compounds, and substituted alcohol and phenol. Particularly, the ophthalmic solution is free of surfactant as well as free of a antimicrobial preservatives defined by the class of quaternary ammonium compounds such as for example, benzalkonium chloride. These classes of compounds are known to have a surfactant effect as well.

In one embodiment, the ophthalmic solution of the present invention consisting essentially of therapeutically effective amount of a prostaglandin esters or amides, cosolvent(s) and self preserving systems and optionally, pharmaceutically acceptable excipients selected from the group consisting of viscosity enhancing agents and buffers. Examples of the self preserving systems are used in the ophthalmic solution of the present invention are Polyquad®, disappearing preservatives include stabilized hydrogen peroxide, stabilized oxy-chlorocomplex, sodium perborate, borate-polyol complex and like.

Therefore, the present invention may be further described as an ophthalmic solution consisting essentially of therapeutically effective amount of a prostaglandin or its analog and, cosolvent(s) and self preserving systems and optionally, pharmaceutically acceptable excipients selected from the group consisting of viscosity enhancing agents and buffers. Since the quaternary ammonium compounds are known to exhibit surfactant activity, the term ‘consisting essentially of’ means that the ophthalmic solution is free of preservatives, particularly, quaternary ammonium preservatives such as Benzalkonium Chloride (BAK), Benzethonium Chloride, Benzyl Alcohol, Busan, Cetrimide, Chlorhexidine, Chlorobutanol, Mercurial Preservatives, or phenylmercuric Nitrate, Phenylmercuric Acetate, Thimerosal, phenylethyl Alcohol and like. However, the safer preservative systems and preservative efficacy enhancers such as edetate disodium, borates, pyruvates, parabens, stabilized oxychloro compounds, Sorbic Acid/Potassium Sorbate Polyaminopropyl Biguanide, Polyquaternium-1, Polyhexamethylene biguanide (PHMB), PVP-Iodine complex, metal ions, peroxides, aminoacids, arginine, tromethamine and mixtures thereof may be included within the scope of the present invention. These compounds are generally regarded as safe and are recommended for long term use.

In certain embodiments of the present invention, another active ingredient may be included in the ophthalmic solution. The another active ingredient that may be included in the ophthalmic solution of the present invention, may be a beta-adrenergic blocking agent which is selected from the group consisting of timolol maleate, betaxalol, levobunolol hydrochloride and their therapeutically active salts or esters. The most commonly used and first line drug for the treatment of glaucoma is timolol maleate. Timolol, a non-selective beta-adrenergic blocking agent, when applied topically as an ophthalmic solution, reduces the intraocular pressure in the eye. It is thus indicated in patients with ocular hypertension or open angle glaucoma. It also shows certain systemic effects which includes (1) beta-adrenergic blockade in the heart causing reduction in cardiac output in both healthy subjects and patients with heart disease and (2) beta-adrenergic receptor blockade in the bronchi and bronchioles resulting in increased airway resistance from unopposed parasympathetic activity. Therefore, the drug must be used with caution in patients in whom beta-adrenergic blockade may be undesirable. Timolol for glaucoma therapy is thus contraindicated in patients with compromised pulmonary functions and in patients who cannot tolerate its systemic cardiovascular action. Hence it is also desirable to reduce the frequency of the use of Timolol maleate wherever possible, preferably as a solution that provides once-a-day administration. Timolol maleate is used in the solutions of the present invention in therapeutically effective amounts. Timolol maleate may be used in an amount ranging from about 0.01% w/v to about 2.0% w/v by weight of the solution, preferably from about 0.05% w/v to about 1.0% w/v by weight of the solution and most preferably from about 0.1% w/v to about 0.5% w/v by weight of the solution. Other beta-adrenergic blocking agent, that is suitable for the present invention is levobunalol or its pharmaceutically acceptable salt. It is used in therapeutically effective amounts 0.5%. In another embodiment, betaxolol or its pharmaceutically acceptable salt is used in amounts ranging from 0.1% w/v to 0.8% w/v, preferably, 0.5% w/v of the ophthalmic solution of the present invention. The preferred amount of beta-adrenergic blocking agent may be included in the concentration of 0.1% w/v to 0.7% w/v, preferably from 0.25% w/v to 0.5% w/v.

The ophthalmic solution of the present invention comprises one or more water soluble excipients selected from a group consisting of a water soluble polymer and a penetration enhancer and mixtures thereof. Examples of the water soluble polymers that may be used in the ophthalmic solution of the present invention, include, but are not limited to, polymers-natural and synthetic, polysaccharides, polyaminoglycosides, cellulose derivatives, guar gum, xanthan gum, geltrite, dextran, hyaluroante, chondroitin sulfate, locust bean gum, polyvinyl alcohol, polyvinyl pyrrolidone, hydroxypropyl methyl cellulose, hydroxyethyl cellulose, carbopol, polystyrene sulfonate and like and mixtures thereof.

The ophthalmic solution of the present invention may further comprise pharmaceutically acceptable excipients conventional to the pharmaceutical art. Typical of such pharmaceutically acceptable excipients include osmotic/tonicity-adjusting agents, one or more pharmaceutically acceptable buffering agents and pH-adjusting agents, viscosity enhancing agents, penetration enhancing vehicles and other agents conventional in art that may be used in formulating an ophthalmic solution or imparting a functional property such as gel-forming, bioadhesion, penetration enhancement and like. In certain embodiments, a combination of two water soluble such as hydroxypropyl methylcellulose and guar gum; hydroxypropyl methylcellulose and a carboxyvinyl polymer; hydroxypropyl methylcellulose and hydroxyethylcellulose; hydroxypropyl methylcellulose and hyaluronic acid; hyaluronic acid and a carboxyvinyl polymer; hyaluronic acid and guar gum; or a carboxyvinyl polymer and guar gum may be incorporated.

The ophthalmic solution of the present invention may be required to be isotonic with respect to the ophthalmic fluids present in the human eye. These solutions are characterized by osmolalities of 250-375 mOsm/kg. Osmolality of the solutions is adjusted by addition of an osmotic/tonicity adjusting agent. Osmotic agents that may be used in the solutions of the present invention to make it isotonic with respect to the ophthalmic fluids present in the human eye, are selected from the group comprising sodium chloride, potassium chloride, calcium chloride, sodium bromide, sodium phosphate sodium sulfate, mannitol, glycerol, sorbitol, propylene glycol, dextrose, sucrose, polyethylene glycols (PEG), PEG-400, PEG-200, PEG300 and the like, and mixtures thereof. In preferred embodiments of the present invention, PEG-400 is used as the osmotic agent. PEG-400 may be present in the solutions of the present invention in an amount ranging from about 1.0% to about 5.0% by weight of the solution, preferably from about 2.5% to about 4.0% by weight of the solution and most preferably in an amount of about 3.0% by weight of the solution.

According to one embodiment, the preservative systems that are considered safer than quaternary ammonium preservatives are preferred such as Polyquad®, stabilized oxy-chlorocomplex, stabilized peroxides and perborates, EDTA, tromethamine, borates, sorbates (such as potassium sorbate and sodium sorbate), parabens (such as methyl-propyl, isopropyl and butyl-paraben) may be used. According to another embodiment of the present invention, the ophthalmic solution may be self preserving. The ingredients that make the solution self preserving includes, but are not limited to, inorganic metal salts such as zinc salts, boric acid, pyruvic acid presence of tromethamine, arginine, histidine, guanidine, disodium edetate or like and mixtures thereof.

In order to achieve, and subsequently maintain, an optimum pH, the ophthalmic solution may contain a pH adjusting agent and/or a buffering agent. The preferred range of pH for an ophthalmic formulation is about 4.0 to about 8.0, and the most preferred pH is about 5.5-7.5. The ophthalmic solution of the present invention comprises a pharmaceutically acceptable pH adjusting agents that may be selected from the group comprising acetic acid or salts thereof, boric acid or salts thereof, phosphoric acid or salts thereof, citric acid or salts thereof, tartaric acid or salts thereof, sodium hydroxide, potassium hydroxide, sodium carbonate, sodium hydrogen carbonate, trometamol, arginine, lysine, histidine, guanine and the like and mixtures thereof. Particularly, preferred pH adjusting agents that may be used in the ophthalmic solution of the present invention include acetic acid, hydrochloric acid, tromethamine, arginine and sodium hydroxide. These agents are used in amounts necessary to produce a pH ranging from about 4.5 to about 8.0.

According to one embodiment the solution of the present invention comprises of one or more solvents or co-solvents. The pharmaceutically acceptable solvents may be selected from a group of alcohols, such as ethanol, glycols such as ethylene glycol, propylene glycol, polyethylene glycol, glycofurol and like.

Besides above mentioned ingredients, one embodiment of the present invention may comprise a number of additional components to provide various functional effects, as is well known in this field. For example, small organic acids may be included as buffers

The present invention also provides a method of treating glaucoma or ocular hypertension which comprises topically administering to an affected eye an ophthalmic solution comprising therapeutically effective amount of a prostaglandin or its analog and a beta-adrenergic blocking agent and water soluble excipient(s) dissolved in a pharmaceutically acceptable vehicle, wherein the solution is free of a surfactant.

In one embodiment, the efficacy of the ophthalmic solution of the present invention was determined by administered the solution to the eyes of the normotensive beagle dogs. The reduction in the intraocular pressure was recorded at time points specifically, 2 hours-which is a time indicator for peak efficacy of a beta-adrenergic blocking agent, 12 hour time point which is a time indicator for peak of therapeutic action for a prostaglandin, and 24 hour time point, being an indicator of the trough level for prostaglandin. Surprisingly, it was found that the method of treating glaucoma or ocular hypertension of the present invention provided improved efficacy in reducing the intraocular pressure when compared to a solution containing a surfactant as such or a preservative that acts like a surfactant within the solution, such as in case of Xalacom® which contains benzalkonium chloride which exerts a surfactant effect apart from acting like a preservative. It is believed by the inventors, without wishing to be bound by any theory, that the surfactant free solution provided improved efficacy because the active agent is directly available on the ocular surface for absorption/partition. It may be postulated that the prostaglandins like latanoprost bind to the micellar core hence less free latanoprost would not be available for absorption/partitioning on the ocular surface. The solution of the present invention is further advantageous in that the ophthalmic solutions having surfactant like BKC or other additives like preservatives cause tearing and eye irritation. Because of which a person skill in the art can expect a significant portion of dose of the active ingredient to be lost. It is also possible that the positively charged benzalkonium chloride resorbs negatively charged latanoprost acid active formed from the pro-drug latanoprost from the ocular surface. This improved effect is evidenced by the data represented in the FIG. I at time points 2 hours or 12 hours. The improved effect is also evidenced at 24 h time point (FIG. I) which is considered trough when the minimum effect is expected.

When the % mean reduction in the intraocular pressure of the affected eye of dogs when the solution of the present invention was administered vs % mean reduction in the intraocular pressure after the administration of marketed reference products such as like Xalatan®, Xalacom®, Timoptic® was studied, it was surprising found that the % mean reduction of the intraocular pressure was higher compared to the marketed product which either contains a beta-adrenergic blocking agent such as Timoptic® or a Xalatan® which is alone or their combination (Xalacom®). It was found that the mean intraocular pressure reduction achieved by administration of the ophthalmic solution of Example 3, was about 34.377% compared to about 26.765% achieved by Xalatan®-latanoprost alone solution or about 28.258% achieved by Xalacom® a combination product of latanoprost and timolol or about 21.088% achieved by Timoptic® which is a timolol alone solution. Please refer to FIG. II.

Surprisingly, it was further found that % reduction in the intraocular pressure when the ophthalmic solution of the present invention was administered when compared with the % reduction in the intraocular pressure after the concomitant administration of marketed latanoprost and Timolol products like Xalatan® and Timoptic® to the dogs, overall, mean intraocular pressure reduction achieved by the ophthalmic solution of the present invention was about 28.63% compared to about 26.49% which was achieved by the concomitant administration of the marketed product of latanoprost (once a day) and Timolol (twice a day) present alone in the products. Concomitant administration may not be desirable due to patient compliance problems and the possible side effects due to higher number of timolol doses.

In another embodiment of the present invention, the IOP reduction from the solution of present invention is more or non-inferior than the reference solutions of latanoprost and Timolol alone or as a fixed dose combination containing a surfactant such as BKC. The IOP reduction was said to be more or non-inferior when at least 50% of the time point at which the IOP readings are taken through out the treatment period show higher or equivalent mean IOP reduction.

Since the solution of the present invention, relates to combination of two active ingredient which vary in their solubility, dose etc. it is important to derive a pharmaceutical vehicle that can incorporate both the actives, particularly without the use of any surfactant, without facing any processing issues, such as drug loss due to incomplete solubilization, precipitation. Thus, in one preferred embodiment, the ophthalmic solution comprises non aqueous solvents such as ethanol, sorbitol, propylene glycol, polyethylene glycol and the like and mixtures thereof. In one embodiment, when the solution is prepared without the application of heat to dissolve the prostaglandin or its derivatives in absence of surfactant, the use of the non aqueous solvents was found to be particularly beneficial, in that the prostaglandin or its derivatives.

One embodiment of the present invention further provides a process of preparation of an ophthalmic solution wherein the solution comprises a polymeric vehicle. In one embodiment, the solution is prepared on a large scale batch such as more beta-adrenergic blocking agent is dissolved in a pharmaceutical vehicle, and preparing the polymeric vehicle separately. The polymeric material in the powder form should be slowly added into the vortex of vigorously agitated water for injection. This process of preparation of polymeric vehicle may be carried out at elevated temperature depending upon the type and nature of the polymer. The solution may be slowly stirred to dissolve the swollen or gelatinized particles completely. Once the water soluble excipient such as the polymeric vehicle is prepared, the active ingredient phase is prepared that is, timolol maleate is separately dissolved in water for injection. Separately, one or more buffering agents such as boric acid may be added and dissolved in the above solution under stirring. Similarly, self preservative agents such as zinc chloride and pH adjusting agents tromethamine are added and dissolved to above solution under stirring. Separately, the prostaglandin derivative such as for example, Latanoprost is taken in a non aqueous solvent such as polyethylene glycol 400 and stirred. This non aqueous solution is added to the timolol maleate aqueous solution under stirring. Since the latanoprost dose is very low, any solution which contains such a low dose drug needs to be done very carefully and with lot of precision. The solution is then filtered. The volume is made up to 20 L with aseptically filtered water for injection and stirred for 30 minutes. The pH is monitored and adjusted to 5.7-6.3, if required. Preferable the pH adjustment step is not carried out. Again the solution is filtered aseptically through 2-20 μm glass fiber disc filter. This step is termed as polishing to make a homogenous polymer solution without the presence of fish-eye type gel particles of polymer. The solution is then filled into containers and the containers are subsequently sealed. The container may be purged with nitrogen.

In one embodiment, the process for the preparation of the ophthalmic solution of the present invention comprises:
a. Preparation of the sterile polymer phase by autoclaving
b. Preparation of the sterile drug phase by aseptic filtration
c. Combining the two phases under aseptic conditions.
d. Optionally, polishing by filtration though 2 micron to 75 micron filter
e. Filling and packaging in eye drop dispensing containers.

In one embodiment, the process for the preparation of the ophthalmic solution of the present invention comprises:
a. Making a prostaglandin phase in a non-aqueous solvent.
b. Adding non-aqueous prostaglandin phase into an aqueous beta-adrenergic blocking agent solution slowly and gradually with stirring
c. Preparation of the sterile polymer phase by autoclaving
d. Combining the two phases under aseptic conditions.
e. Optionally, polishing by filtration though 2 micron to 75 micron filter to remove foreign particulates
f. Filling and packaging in eye drop dispensing containers.

While the present invention is disclosed generally above, additional aspects are further discussed and illustrated with reference to the examples below. However, the examples are presented merely to illustrate the invention and should not be considered as limitations thereto.

### Example 1-2

TABLE 1
Composition of the ophthalmic solution

| | | Example 1 | Example 2 |
|---|---|---|---|
| S. No | Ingredients | Qty (% w/v) | |
| 1. | Latanoprost | 0.0025 | 0.005 |
| 2. | Polyethylene glycol 400 | 3.0 | 3.0 |
| 3. | Hydroxypropyl methylcellulose | — | 0.5 |
| 4. | polyvinyl pyrrolidone | — | 2.0 |
| 5. | Boric acid | 1.0 | 1.0 |
| 6. | Zinc Chloride | 0.0025 | 0.0025 |
| 7. | Tromethamine | 0.375 | 0.375 |
| 8. | Water for injection | qs | qs |

The ophthalmic solution according to example 1 and 2 are prepared by the procedure.

The ophthalmic solutions of Example 1, was stored in parylene coated containers as well as uncoated LDPE containers. Surprisingly, it was found that the solution remained stable in terms of chemical assay when stored in parylene coated bottles.

TABLE 2
Stability results of the ophthalmic solution of Example 1
Stability data

| Assay of Latanoprost in | | | Assay of Latanoprost in Uncoated | | |
|---|---|---|---|---|---|
| Parylene coated bottles | | | LDPE containers | | |
| Initial | 1D/85° C. | 3D/60° C. | Initial | 1D/85° C. | 3D/60° C. |
| 106.15 | 97.02 | 98.01 | 103.21 | 58.76 | 71.38 |

Further, the chemically stable ophthalmic solution of Example 1 was tested for efficacy in six beagle dogs for its antihypertensive action. The duration of the study was 10 days. 30 microlitres of the solution of Example 1 which contains 25 ng/μl of latanoprost was instilled into the eye of the beagle dogs. The measurement of reduction in intraocular pressure was recorded at initial 12 hour and 24 hour time points. The results of the efficacy study are tabulated in Table 3 as follows:

TABLE 3
Results of the efficacy of the ophthalmic solution of latanoprost as per
Example 1 that is free of surfactant in comparison to marketed product,
Xalatan ® which contains benzalkonium chloride, a surfactant

| | | | Average | |
|---|---|---|---|---|
| | Concentration | | % IOP | Ratio |
| | of | | reduction | of % IOP |
| | latanoprost | Dose instilled | at 12 hour | reduction per |
| Test | (ng/microlitre) | (micrograms) | time point | microgram |
| Example 1 | 25 ng/μl | 0.75 | 27.43 ± 6.23 | 36.57 |
| Xalatan ® | 50 ng/μl | 1.5 | 29.86 ± 5.33 | 19.90 |

It may be concluded from the Table 3, that the ophthalmic solution of present invention which is free of surfactant, when administered at half the dose compared to the Xalatan®, the solution achieved almost equivalent efficacy in terms of intraocular pressure reduction. Thus, there is a surprising effect of achievement of equivalent efficacy at half the dose of latanoprost. This effect is indeed surprising and unexpected. Further, only half of the latanoprost dose present in the ophthalmic solution of the present invention compared to Xalatan®, was found to provide reduction in the intraocular pressure at time points of 6 hours, 12 hours and 24 hours. Unexpectedly, it was further found that the % intraocular pressure reduction at 12 hour time point is higher compared to the % intraocular pressure reduction at 12 hours, for Xalatan®.

### Example 3

TABLE 4
Ophthalmic solution of the present invention

| S. No | ingredients | Qty (% w/v) |
|---|---|---|
| 1. | Timolol Maleate eq to Timolol | 0.50 |
| 2. | Latanoprost | 0.005 |
| 3. | Polyethyleneglycol 400 | 3.0 |
| 4. | Hypromellose 2910 | 0.5 |
| 5. | PVP K 90 | 2.0 |
| 6. | Boric acid | 1.0 |
| 7. | Zinc Chloride | 0.0025 |
| 8. | Tromethamine | 0.375 |
| 9. | Water for injection | qs |

The solution was prepared as described in the description text without the use of any surfactant. The transmittance of the final solution was found to be 98.45%. The % transmission when stored at varying conditions for one month showed the following values. Also, the solution was found to be stable when stored in parylene coated containers as compared to the uncoated LDPE containers as shown in table 4.

TABLE 5
Stability data

| | Assay of Latanoprost (% of label Claim) at | | | | |
|---|---|---|---|---|---|
| | different storage conditions | | | | |
| | Initial and 1 month at varying storage conditions | | | | |
| Storage | | | 25° C./ | 30° C./ | 40° C./ |
| container | Initial | 2-8° C. | 40% RH | 35% RH | 25% RH |
| Uncoated | 99.32 | 94.89 | 87.45 | 82.12 | 77.60 |
| LDPE | | | | | |
| container | | | | | |
| Parylene | 98.89 | 100.93 | 101.20 | 101.69 | 101.08 |
| Coated | | | | | |
| container | | | | | |
| Clarity | | | | | |
| on storage | % Transmission | | | | |
| Solution of | 98.5 | 99.3 | 99.2 | 98.6 | 99.7 |
| example 3 | | | | | |

Although there was no potency loss of active ingredient when the solution was kept in coated bottles for one month in stability, however significant potency loss of drug substance was observed in uncoated LDPE plastic bottles. This indicates that Parylene coating can prevent the absorption/adsorption of drug substance on to the LDPE plastic containers.

### Example 4

The ophthalmic solution of the present invention which is surfactant free, and preferably, substantially free of preservative, was subjected to antimicrobial Effectiveness Test as per USP/JP. The results are documented in Table 6 below.

TABLE 6
Results of antimicrobial test as per USP/JP monograph

| Acceptance Criteria as per USP | | |
|---|---|---|
| monograph | Organism | Observation |
| NLT 1.0 log reduction from initial | Escherichia coli. | Complies |
| count at 7 days; NLT 3.0 log reduction | Pseudomonas | |
| from initial count at 14 days and no | aeruginosa | |
| increase from the 14 days count at 28 | Staphylococcus | |
| days. | aureus | |
| No increase** from the initial | Candida albicans | Complies |
| calculated count at 7, 14, and 28 days | Aspergillus Niger | |

It may be concluded that the ophthalmic solution of the present invention, passes the compendial antimicrobial effectiveness testing criteria.

### Example 5

TABLE 7
Composition of the ophthalmic solution

| S. No | ingredients | Qty (% w/v) |
|---|---|---|
| 1. | Timolol Maleate eq to | 0.50 |
| | Timolol | |
| 2. | Travoprost | 0.004 |
| 3. | Polyethyleneglycol 400 | 3.0 |
| 4. | Hypromellose 2910 | 0.5 |
| 5. | PVP K 90 | 2.0 |
| 6. | Boric acid | 1.0 |
| 7. | Zinc Chloride | 0.0025 |
| 8. | Tromethamine | q.s. |
| 9. | WFI | q.s. |

The ophthalmic solution according to the constituents Example 5 was prepared by a process similar to Example 3, except, latanoprost was substituted by travoprost. The pH was adjusted to 6.0. The % Transmittance was found to be 98.913.

### Example 6

TABLE 8
Composition of the ophthalmic solution

| Sl. No | Ingredients | Qty (% w/v) |
|---|---|---|
| 1. | Betaxolol Hydrochloride eq to | 0.50 |
| | Betaxolol | |
| 2. | Latanoprost | 0.005 |
| 3. | Polyethyleneglycol 400 | 3.0 |
| 4. | Hypromellose 2910 | 0.5 |
| 5. | PVP K 90 | 2.0 |
| 6. | Boric acid | 1.0 |
| 7. | Zinc Chloride | 0.0025 |
| 8. | Tromethamine | qs |
| 9. | Water for injection | qs |

The ophthalmic solution according to the constituents Example 6 was prepared by a process similar to Example 3, except, timolol maleate was substituted by Betaxolol another beta-adrenergic blocking agent. The pH was adjusted to 6.0. The % Transmittance was found to be 96.473.

### Example 7

TABLE 9
Composition of the ophthalmic solution

| Sl. No | ingredients | Qty (% w/v) |
|---|---|---|
| 1. | Betaxolol Hydrochloride eq | 0.50 |
| | to Betaxolol | |
| 2. | Travoprost | 0.004 |
| 3. | Polyethyleneglycol 400 | 3.0 |
| 4. | Hypromellose 2910 | 0.5 |
| 5. | PVP K 90 | 2.0 |
| 6. | Boric acid | 1.0 |
| 7. | Zinc Chloride | 0.0025 |
| 8. | Tromethamine | q.s. |
| 9. | WFI | q.s. |

The ophthalmic solution according to the constituents Example 7 was prepared by a process similar to Example 3, except, timolol maleate was substituted by Betaxolol another beta-adrenergic blocking agent and latanoprost was substituted by travoprost. The pH was adjusted to 6.0. The % Transmittance was found to be 98.266.

### Example 8

The solution prepared according to example 3 was subjected to a comparative efficacy study in normotensive beagle dogs. The efficacy was compared with three marketed reference formulations namely, (Xalacom®, Xalatan® and Timoptic®) which contains latanoprost and timolol Maleate in combination; latanoprost alone and timolol Maleate alone, respectively.

Three healthy beagle dogs were taken for each group. Pretreatment measurement of intraocular pressure were obtained for both eyes at 8.00 AM and 8.00 PM for 2 days preceding treatment with the help of 30 Classic Pneumatonometer Model 30 (Reichert, USA) and considered as initial intraocular pressure reading. 30 μl of solution of example 3, Xalacom and Xalatan were instilled in the treated eyes once a day at 8 am whereas 30 μl of Timoptic was instilled in the treated eyes two times a day at 8 am and 8 pm on day 3 to day 12. On day 3 IOP was measured at 2, 6, 12 and 24 h after medicament instillation and from day 4 to day 12 the IOP was measured at 2, 12 and 24 h after dosing. After the treatment period, on day 13 to day 17 IOP measurements were obtained once each day at 9.00 am.

A comparative % reduction in the intraocular pressure of the dogs within 24 hours when the solution of the present invention was administered and % reduction in the intraocular pressure after the administration of already available marketed products like Xalatan®, Xalacom®, Timoptic® was calculated.

For representation purposes, the reduction in the intraocular pressure was plotted at 2 hours, 12 hours and 24 hours time points and is plotted as provided in FIG. I. During the first 24 h when the treated eyes were first exposed to the medicaments, the IOP reduction of solution of present invention was more than other marketed formulations such as Xalatan®, Xalacom® and Timoptic®. Further it was observed that throughout the treatment period, the intraocular pressure reduction by administration of the solution of the present invention was higher in comparison to the marketed formulations.

### Example 9

The solution prepared according to example 3 was subjected to a comparative efficacy study in normotensive beagle dogs. The efficacy was compared with marketed reference formulations namely, Xalatan® and Timoptic® which contain latanoprost and timolol Maleate, respectively, was co-administered. Pretreatment measurement of IOP was obtained for both the eyes of each beagle dogs at 8 am and 8 pm for two days preceding treatment (day 1 to day 2). On day 3 animals were divided into 2 groups consisting of 6 animals. One group of animals received 30-μL instillation of Test (example 3 of the present invention) to one eye once daily and another group received 30-μL Xalatan® once daily and 30-μL Timoptic® instilled twice daily in same eye received for 10 days and IOP readings were measured, as described above. Almost equivalent or slightly improved efficacy was found when ophthalmic solution of the present invention was compared to concomitant administration of Xalatan® and Timoptic® (FIG. III).

### Comparative Example 1

TABLE 10

| Sr. No | ingredients | Qty (% w/v) |
|---|---|---|
| 1. | Timolol Maleate eq to Timolol | 0.50 |
| 2. | Latanoprost | 0.005 |
| 3. | Castor oil | 0.15 |
| 4. | Solutol HS 15 | 0.25 |
| 5. | HPMC | 0.5 |
| 6. | PVP K 90 | 2.0 |
| 7. | Boric acid | 1.0 |
| 8. | Polyethylene glycol | 3.0 |
| 9. | Zinc Chloride | 0.0025 |
| 10. | Tromethamine | 0.375 |
| 11. | Water for Injection | q.s. |

| | |
|---|---|
| pH | 6.5-7.5 |

### Procedure:

1. Collect Water for Injection (WFI) of temperature between 20 to 25° C. in a vessel. Add and dissolve Boric acid, sodium borate/Borax, Edetate disodium, potassium sorbate and timolol maleate with continuous stirring. Ensure complete solubilisation of all the ingredients added above and clarity of solution visually.
2. Take Latanoprost and castor oil in a glass beaker. Stir it with glass rod. Take Macrogol 15 Hydroxystearate in a separate beaker and heat it at 65-70° C. After melting, transfer it to the above oil phase. Stir using dry glass rod at 65-70° C. Maintain the temperature at 65-70° C. with heating.
3. Take WFI and heat it at 70-75° C. in a vessel fitted with silverson homogenizer. Take additional small quantity of WFI and heat it at 70-75° C. in another 316 vessel and maintain the temperature between 70-75° C. until use.
4. Add the Oil phase drop wise to WFI at 70-75° C. under high speed stirring.
5. Rinse the containers used for oil phase and Macrogol-15-Hydroxystearate with additional pre-heated WFI and add to the above solution at 70-75° C. under high speed stirring. Continued the high speed stirring for 10 min. Reduce the speed. Bring down the temperature. Add propylene glycol under mild stirring.
6. Add the Timolol solution prepared at step 1 to the solution under stirring.

### 7. Check pH.

8. Make up the volume with WFI.

The % transmittance was recorded as per the description. It was found to be only 2.19%.

### Comparative Example 2

TABLE 11

| Sr. No | ingredients | Qty (% w/v) |
|---|---|---|
| 1. | Timolol Maleate eq to Timolol | 0.50 |
| 2. | Latanoprost | 0.005 |
| 3. | Castor oil | 0.10 |
| 4. | Solutol HS 15 | 0.25 |
| 5. | HPMC | 0.5 |
| 6. | PVP K 90 | 2.0 |
| 7. | Boric acid | 1.0 |
| 8. | Sodium chloride | 0.65 |
| 9. | Zinc Chloride | 0.0025 |
| 10. | Tromethamine | q.s. |
| 11. | Water for Injection | q.s. |

| | |
|---|---|
| pH | 6.5-7.5 |

The comparative example 2 was prepared as per the procedure followed for preparing comparative example 1. The comparative example 2 is different than the comparative example 1 in that it contains reduced amount of the castor oil compared to the comparative example 1. The solution so prepared was checked for the % transmittance. The % transmittance was found to be 79.1 at initial point and when stored for 6 months at 2-8° C. it was found to be to 65.7.

Thus, it could be concluded that the incorporation of an oil along with surfactant into the solution of combination of a prostaglandin and a beta-adrenergic blocking agent, do not provide a clear solution.

1. An ophthalmic solution comprising therapeutically effective amount of a prostaglandin or its analog and water soluble excipient(s) dissolved in a pharmaceutically acceptable vehicle, wherein the solution is free of a surfactant.

2. An ophthalmic solution as claimed in claim 1 further comprises a beta adrenergic blocking agent.

3. An ophthalmic solution as claimed in claim 1 wherein water soluble excipient(s) are water soluble polymer or one or more penetration enhancing agents.

4. An ophthalmic solution as claimed in claim 1 wherein the solution is free of preservatives which are organic mercurial compounds, quaternary ammonium compound or substituted alcohol or phenol.

5. An ophthalmic solution as claimed in claim 3 wherein the solution is stored in a parylene coated plastic bottle.

6. An ophthalmic solution consisting essentially of therapeutically effective amount of a prostaglandin or its analog, co-solvent(s) and self preserving system and optionally, pharmaceutically acceptable excipients selected from the group consisting of viscosity enhancing agents and buffers.

7. An ophthalmic solution as claimed in claim 6 wherein viscosity enhancing agents is a water soluble polymer.

8. A method of treating glaucoma or ocular hypertension which comprises topically administering to an affected eye an ophthalmic solution defined by claim 1.

9. A method of treating glaucoma or ocular hypertension which comprises topically administering to an affected eye an ophthalmic solution defined by claim 2.

10. A method of treating glaucoma or ocular hypertension which comprises topically administering to an affected eye an ophthalmic solution defined by claim 3.

11. A method of treating glaucoma or ocular hypertension which comprises topically administering to an affected eye an ophthalmic solution defined by claim 4.

12. A method of treating glaucoma or ocular hypertension which comprises topically administering to an affected eye an ophthalmic solution defined by claim 5.

13. A method of treating glaucoma or ocular hypertension which comprises topically administering to an affected eye an ophthalmic solution defined by claim 6.

14. A method of treating glaucoma or ocular hypertension which comprises topically administering to an affected eye an ophthalmic solution defined by claim 7.
